(19) United States
(12) Patent Application Publication
(10) Pub. No.: US 2007/0167971 A1
(43) Pub. Date: Jul. 19, 2007

(54) Devices and methods for promoting the formation of blood clots in esophageal varices

(76) Inventors: Raymond Huey, Orange, CT (US); Jeffrey L. Horn, Rocky Hill, CT (US); Denny Lo, Bethlehem, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/654,409

(22) Filed: Jan. 17, 2007

Related U.S. Application Data
(60) Provisional application No. 60/759,775, filed on Jan. 17, 2006.
(60) Provisional application No. 60/808,618, filed on May 26, 2006.
(60) Provisional application No. 60/810,447, filed on Jun. 1, 2006.

(51) Int. Cl. A61M 29/00

(52) U.S. Cl. 606/191

(57) ABSTRACT

A device for promoting the clotting of blood in body cavities includes a flexible body portion; an expandable member located on the flexible body portion; and a blood clotting material attached to the expandable member. When used, insertion of at least a portion of the blood clotting material into the body cavity causes at least a portion of the blood clotting material to contact blood emanating from a bleed site. Methods of providing therapies to tube-shaped organs include the steps of providing suitable devices having expansion capabilities, positioning the devices at the appropriate bleed sites, and expanding the devices to cause blood clotting materials to contact the bleed sites. Materials that may be used as the blood clotting material include zeolites, molecular sieve materials, diatomaceous earth, clay, silica-based materials, oxidized cellulose, carboxymethyl cellulose, bioactive glass, biological hemostats, chitosan, and combinations of the foregoing.

### CROSS REFERENCE TO RELATED APPLICATIONS

This application is related to and claims the benefit of U.S. Provisional Patent Application No. 60/759,775 filed on Jan. 17, 2006, the contents of which are incorporated by reference herein in their entirety. This application is also related to U.S. Provisional Patent Application Ser. No. 60/808,618, filed May 26, 2006, entitled “Blood Clotting Compound”; U.S. Provisional Patent Application Ser. No. 60/810,447, filed Jun. 1, 2006, entitled “Hemostatic Device with Oxidized Cellulose Pad”; U.S. patent application Ser. No. 11/544,238, filed Oct. 6, 2006, entitled “Hemostatic Compositions and Method of Manufacture”; U.S. patent application Ser. No. 11/584,079, filed Oct. 20, 2006, entitled “Devices and Methods for the Delivery of Hemostatic Agents to Bleeding Wounds”; U.S. patent application Ser. No. 11/590,427, filed Oct. 30, 2006, entitled “Clay-Based Hemostatic Agents and Devices for the Delivery Thereof”; U.S. patent application Ser. No. ______ Attorney Docket No. 6989-0073, filed Nov. 29, 2006, entitled “Heat Mitigating Hemostatic Agent”; and U.S. patent application Ser. No. 11/633,687, filed Dec. 4, 2006, entitled “Hemostatic Agents and Devices for the Delivery Thereof”; the contents of all of the above-referenced applications being incorporated herein by reference in their entireties.

### TECHNICAL FIELD

The present invention relates generally to blood clotting devices and, more particularly, to blood clotting materials, devices incorporating such materials, and methods for the delivery of such materials for use in controlling bleeding in esophageal varices.

### BACKGROUND OF THE INVENTION

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding (minor discharges of blood) or hemorrhaging (discharges of copious amounts of blood) occurs, the fibrinogen reacts with water and thrombin (an enzyme) to form fibrin, which is insoluble in blood and polymerizes to form clots.

The esophagus is an elongated organ that carries food and liquids from the throat into the stomach. Located immediately behind the trachea, the esophagus is defined by a tube-shaped muscular wall. Upon swallowing, the muscles of the wall contract to push food down in the direction of the stomach. Glands in the esophagus lining produce mucus to moisten the inner wall of the esophagus, thereby aiding in the swallowing action and facilitating the passage of food. A sphincter is located at the junction of the esophagus and the stomach. As the swallowed food approaches the sphincter, the muscles of the lower esophagus relax and the sphincter opens to allow the food to pass into the stomach.

Esophageal varices can form in the esophagus. These varices are enlarged or swollen veins on the lining of the esophagus wall. Typically, the varices form at the lower end of the esophagus proximate the sphincter. Although they can appear for a variety of reasons, the primary reason for the formation of esophageal varices is liver disease, e.g., liver cirrhosis. Liver disease is generally the cause of portal hypertension, which is increased blood pressure in the portal vein. At higher blood pressures, the vessels in the esophagus that are in fluid communication with the vein as well as the vein itself may rupture and pass blood into the esophagus. Smaller amounts of blood are generally passed through the sphincter into the stomach, while larger amounts of blood resulting from hemorrhaging are vomited.

One known method of treating esophageal bleeding is sclerotherapy, which involves injecting a sclerosant into the ruptured veins and surrounding area. Typical sclerosants, which are known to promote the clotting of blood, include ethanolamine and sodium tetradecyl sulfates, both of which may be irritating to the tissue of the esophageal wall. A local anesthetic such as hyoscine butylbromide may be administered to freeze the tissue of the esophagus wall to facilitate the injection of the sclerosant. Generally, sclerotherapy is endoscopic, which means an endoscope is passed through the oral cavity of the patient to the esophagus to enable the physician to view the bleeding sites and the administered therapy.

One problem with sclerotherapy and similar methods of the prior art is that discomfort is caused to the patient. In particular, the intramuscular injection of sclerosant into the esophagus wall may be painful. Moreover, the administration of a local anesthetic to freeze the tissue generally causes some amount of discomfort. Accordingly, there is a need for an improved clotting device and method of its use that can quickly stop the bleeding or hemorrhaging associated with esophageal varices.

Based on the foregoing, it is a general object of the present invention to provide devices for controlling esophageal bleeding or hemorrhaging and methods of their use that overcome the problems with or improve upon the prior art.

### SUMMARY OF THE INVENTION

According to one aspect, the present invention resides in a device for promoting the clotting of blood in body cavities. The device includes a flexible body portion; an expandable member located on the flexible body portion; and a blood clotting material attached to the expandable member. When such a device is used to treat a bleeding wound, insertion of at least a portion of the blood clotting material into the body cavity causes at least a portion of the inserted blood clotting material to contact blood emanating from a bleed site. When the blood clotting material contacts the blood at the bleed site, it reacts thereto causing clotting to occur.

In another aspect, the present invention resides in a device employed to facilitate the clotting of blood in which the blood clotting material is disposed on a substrate that is attached to or forms part of an expandable member. The substrate may be any one of a variety of materials, e.g., paper, polymer, foam, and the like, however, the present invention is not limited in this regard as other suitable materials known to those skilled in the pertinent art to which the present invention pertains may be substituted without departing from the broader aspects of the present invention. Furthermore, the substrate may be a synthetic material or a non-synthetic material.

In other aspects, the present invention resides in blood clotting devices in which the expandable member is a balloon or a bellows, expandable using a pressurized fluid such as air or liquid. The present invention further resides in a device in which the expandable member is made from a shape memory alloy, the actuation of which causes the expandable member to assume a predetermined shape.

In yet other aspects, the present invention resides in methods of providing therapies to tube-shaped organs (for example, an esophagus or a colon or the like) by clotting blood emanating from bleed sites. Such methods comprise the steps of providing suitable devices having expansion capabilities, positioning the devices at the appropriate bleed sites, and expanding the devices to cause blood clotting materials to contact the bleed sites.

A blood clotting material found to be particularly effective in causing blood to clot is zeolite. The zeolite is attached to, incorporated into, or impregnated into the expandable member (e.g., the balloon, the bellows, or the cylinder). Alternately, the zeolite may be attached to, incorporated into, or impregnated into the substrate that is attached to the expandable member. While zeolite has been described, the present invention is not limited in this regard. Other materials that may be used as the blood clotting material include, but are not limited to, molecular sieve materials, diatomaceous earth, clay, silica-based materials, oxidized cellulose, carboxymethyl cellulose and salts thereof, bioactive glass, biological hemostats, chitosan, combinations of the foregoing with or without zeolite, and the like.

In embodiments incorporating zeolite, the zeolite contains less than about 75% by weight silicon oxide, and preferably less than about 65% by weight silicon oxide; less than about 50% by weight aluminum oxide, and preferably less than about 40% by weight of aluminum oxide; less than about 30% by weight sodium oxide, and preferably less than about 20% by weight of sodium oxide; less about 30% by weight of calcium oxide, and preferably less than about 20% by weight of calcium oxide. Preferably, zeolite is impregnated into the substrate which in the preferred embodiment is a paper. While the material has been described as being impregnated into the paper, the present invention is not limited in this regard as the zeolite can also be adhesively attached to the paper without departing from the broader aspects of the invention. In addition, the substrate is not limited to paper as other suitable substrates known to those skilled in the pertinent art to which the present invention pertains, such as polymers or gauze, can also be employed.

The devices and methods of the present invention are especially useful in addressing internal bleeding at esophageal varices. The devices and methods of the present invention are also applicable in other situations, particularly those in which it is desirable to stop internal bleeding at any tubular organ (e.g., in the colon) as well as in nasal passages or the nasal cavity or in the mouth during dental applications. The present invention may also be applicable in situations involving surface area damage to tube-shaped organs damaged during injuries or planned surgical procedures.

One advantage of the present invention is that upon use of any of the devices of the present invention to treat a bleeding internal wound, injection into damaged, tender, bleeding tissue is avoided. Thus, pain and discomfort caused to a patient are minimized or eliminated altogether.

Another advantage of the present invention is that use of the therapies disclosed herein allow a blood clotting material to be applied to a wound and removed upon completion of the treatment of the wound. Such therapies are in contrast to those of the prior art in which blood clotting materials are injected into the tissue and remain there until they are passed naturally out of the body. The nature of the blood clotting material used with the devices and methods of the present invention allows the material to be, in effect, topically applied as needed then removed such that little or no foreign material is left at the site of the treated issue.

Another advantage of the present invention with regard to embodiments incorporating zeolite material is that the particle form of the zeolite allows it to react less exothermically than other forms of zeolite (e.g., powder). The porous nature of the material still allows liquid blood constituents to be wicked away to cause thickening of the blood, thereby facilitating the formation of clots. The initial moisture content of the zeolite can be controlled such that a less aggressive drawing of moisture from the blood is realized, which thereby tempers the exothermic effects experienced at the wound site.

In embodiments in which carboxymethyl cellulose, salts of carboxymethyl cellulose, or combinations thereof are used to address bleeding wounds, the material also functions as a gelling agent. In such embodiments, the gel nature of the material allows for its ease of topical application to a wound. Furthermore, when applied to a wound and covered with a bandage, the gel nature of the material allows for the easy release of the bandage. Moreover, carboxymethyl cellulose and the salts thereof are compatible with living tissue.

Still another advantage of the present invention is that the proper dose of blood clotting material can be readily applied to a wound. Particularly when the device is an expandable and collapsible member on which the zeolite or other blood clotting material is disposed, the device can be readily removed from a sterilized packaging and used immediately. Guesswork, estimation, or calculation of the amounts of blood clotting material for application to a bleeding wound is eliminated since there is a definite amount of material associated with the device. Accordingly, little or no material is wasted.

### BRIEF DESCRIPTION OF THE DRAWINGS

FIG. 1 is a perspective view of a blood clotting device of the present invention in an expanded state.

FIG. 2 is side view of the blood clotting device of FIG. 1 in an expanded state.

FIG. 3 is a side view of the blood clotting device of FIG. 1 having a protective wax coating disposed thereover for insertion.

FIG. 4A is a perspective view of an alternate embodiment of a blood clotting device of the present invention in an unexpanded state.

FIG. 4B is a side view of the blood clotting device of FIG. 4A in an expanded state.

FIG. 5A is a perspective view of another alternate embodiment of a blood clotting device of the present invention in an unexpanded state.

FIG. 5B is a perspective view of the blood clotting device of FIG. 5A in an expanded state.

FIG. 6 is a schematic representation of a highly magnified section of a blood clotting device of the present invention.

FIG. 7 is a side view of the blood clotting device of FIG. 6 illustrating one means of retaining blood clotting material in powder form on a substrate.

### DETAILED DESCRIPTION OF THE PREFERRED EMBODIMENT

Disclosed herein are devices, methods, and systems for delivering materials to internal bleed sites, particularly bleed sites in cavities, and more particularly bleed sites that result from the presence of esophageal varices, to promote the clotting of blood. The devices can be inserted into the esophagus during minimally invasive surgical procedures to cause hemostatic (blood clotting) materials to contact sites from which blood emanates. By maintaining such contact, the blood clotting materials absorb at least portions of the liquid phases of the blood, thereby promoting clotting. The devices of the present invention comprise balloons or other expandable devices on which the blood clotting materials are disposed. The blood clotting material of the present invention is any suitable material capable of causing hemostasis when maintained in contact with blood emanating from a wound.

In one embodiment of the present invention, the blood clotting material is a molecular sieve material. The molecular sieve material used in the present invention may be a synthetic polymer gel, cellulose material, porous silica gel, porous glass, alumina, hydroxyapatite, faujasite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

In at least one embodiment of the present invention, the molecular sieve material is a zeolite. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the zeolite is a friable material that is less than about 75% by weight silicon oxide, and preferably less than about 65% by weight silicon oxide; less than about 50% by weight aluminum oxide, and preferably less than about 40% by weight aluminum oxide; less than about 30% by weight sodium oxide, and preferably less than about 20% by weight of sodium oxide; less than about 30% by weight of calcium oxide, and preferably less than about 20% by weight of calcium oxide. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. The zeolite is in particle form, and the median size of the zeolite particle used is about 7 microns. However, the present invention is not limited in this regard as other sizes of zeolite particle are within the scope of the invention.

The zeolite may be mixed with or otherwise used in conjunction with other materials that can be dehydrated without significant changes in crystalline structure. Such materials include, but are not limited to, magnesium sulfate, sodium metaphosphate, calcium chloride, dextrin, polysaccharides, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides. One zeolite material found to be particularly useful in practicing the present invention is MOLSIV ADSORBENTS 5A, manufactured by UOP LLC of Des Plaines, Ill. However, the present invention is not limited in this regard as other zeolite materials can be substituted without departing from the broader aspects of the present invention.

The present invention is also not limited to the use of zeolites as the blood clotting material, however, as other materials are within the scope of the present invention and can be used in place of or in addition to zeolites. For example, diatomaceous earth can be employed as a blood clotting material. Diatomaceous earth, when brought into contact with a bleeding wound, can minimize or stop blood flow by absorbing at least portions of the liquid phases of the blood, thereby facilitating clotting. The use of other silica-based materials such as clays in conjunction with diatomaceous earth is also within the scope of the present invention.

As used herein, the term “diatomaceous earth” refers to a mineral derived from the fossilized shell remains of fresh water algae and marine algae. These algae are unicellular or colonial algae from the class Bacillariaphyccae and are known as diatoms. Diatoms are characterized by very irregular shapes and generally spiny structures having pitted surface areas. Structurally, they may be barrel-shaped, cylindrical, disk-shaped, etc. and average about 5 to about 20 microns in diameter.

The diatomaceous earth mineral, which is composed of the diatoms and is typically found in deposits in sedimentary rock formed as the result of receding waterlines in lakes and oceans, is about 86% silicon, about 5% sodium, about 3% magnesium, and about 2% iron, such components typically being present in oxide form. Other elements such as copper, strontium, manganese, titanium, and sodium, as well as other elements, may also be found in diatomaceous earth. The porosity of diatomaceous earth is about 85%.

Other exemplary materials that may be used to provide hemostasis include clay materials or other silica-based materials that, when brought into contact with a bleeding wound, can minimize or stop blood flow by absorbing at least portions of the liquid phases of the blood, thereby facilitating clotting. As used herein, the term “clay” refers to a crystalline form of hydrated aluminum silicate. The crystals of clay are irregularly shaped and insoluble in water. The combination of some types of clay with water may produce a mass having some degree of plasticity. Depending upon the type of clay, the combination thereof with water may produce a colloidal gel having thixotropic properties.

In one preferred embodiment of the present invention, the clay material is kaolin, which includes the mineral “kaolinite.” Although the term “kaolin” is used hereinafter, it should be understood that kaolinite may also be used in conjunction with or in place of kaolin. The present invention is also not limited with regard to kaolin or kaolinite, as other materials are within the scope of the present invention. Such materials include, but are not limited to, attapulgite, bentonite, combinations of the foregoing, combinations of the foregoing with kaolin and/or diatomaceous earth, and/or zeolite, and the like.

As used herein, the term “kaolin” refers to a soft, earthy aluminosilicate clay (and, more specifically, to a dioctahedral phyllosilicate clay) having the chemical formula Al₂Si₂O₅(OH)₄. Kaolin is a naturally occurring layered silicate mineral having alternating tetrahedral sheets and octahedral sheets of alumina octahedra linked via the oxygen atoms of hydroxyl groups. Kaolin comprises about 50% alumina, about 50% silica, and trace impurities.

More preferably, the clay is Edgar's plastic kaolin (hereinafter “EPK”), which is a water-washed kaolin clay that is mined and processed in and near Edgar, Fla. Edgar's plastic kaolin has desirable plasticity characteristics, is castable, and when mixed with water produces a thixotropic slurry.

The kaolin material of the present invention may be mixed with or otherwise used in conjunction with other materials to provide additional clotting functions and/or improved efficacy. Such materials include, but are not limited to, magnesium sulfate, sodium metaphosphate, calcium chloride, dextrin, combinations of the foregoing materials, and hydrates of the foregoing materials.

Another exemplary material that may be used to provide blood clotting functions includes oxidized cellulose. Oxidized cellulose is a chemically oxidized form of a common cellulose fiber such as cotton and is also known as cellulosic acid, absorbable cellulose, or polyanhydroglucuronic acid. The degree of oxidation of the fiber is a function of the carboxylation content of the fibrous cellulose material. In particular, as the number of carboxyl groups on the cellulose structure is increased, the oxidation content correspondingly increases.

Oxidized cellulose may be manufactured by the action of nitrogen dioxide gas (NO₂) on cellulose fiber. Other methods of manufacturing oxidized cellulose include oxidation of cellulose fiber with aqueous oxidizing agents such as hypochlorite salts, although the use of such agents is less preferred than the use of nitrogen dioxide gas.

Irrespective of its method of manufacture, the oxidized cellulose is typically a mass of unwoven cellulose strands that are loosely intermingled and easily compressed. The interstices between adjacent strands define areas in which the blood collects and the solids thereof agglomerate to facilitate the formation of clots. The compressibility of the unwoven cellulose strand mass allows the material to be formed into sheets from which pellets, particles, beads, or the like can be cut, stamped, or otherwise formed.

Other exemplary materials that may be used to provide blood clotting functions include carboxymethyl cellulose, salts of carboxymethyl cellulose, and combinations of the foregoing. Carboxymethyl cellulose is a water-soluble polymer in which carboxylic acid groups are substituted on the glucose units of a cellulose chain through an ether linkage (R—O—CH₂COOH, where R is the glucose unit). In producing carboxymethyl cellulose, the functional groups are generally the sodium salts of the carboxylic acid (R—O—CH₂COONa). The present invention is not limited to the sodium salt of carboxylic acid, however, as other salts of carboxylic acid are within the scope of the present invention.

Other materials such as bioactive glasses, biological hemostats, chitosan, combinations of the foregoing, and the like are within the scope of the present invention and can be used either separately as blood clotting materials or in conjunction with the zeolite, diatomaceous earth, clay, oxidized cellulose, carboxymethyl cellulose, or salts of carboxymethyl cellulose. Biological hemostats include, but are not limited to, absorbable gelatins, collagen, and the like.

Various materials may be mixed with, associated with, or incorporated into the zeolites, diatomaceous earth, clay, oxidized cellulose, carboxymethyl cellulose, salts of carboxymethyl cellulose, bioactive glass, biological hemostat, chitosan, or other material to maintain an antiseptic environment at the wound site or to provide functions that are supplemental to the clotting functions of the blood clotting materials. Exemplary materials that can be used include, but are not limited to, pharmaceutically-active compositions such as antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), bacteriostatics, compounds containing copper ions and/or silver ions, wound healing agents, and the like. Still other materials that can be incorporated to provide additional hemostatic functions include ascorbic acid, tranexamic acid, rutin, and thrombin. Botanical agents having desirable effects on the wound site may also be added.

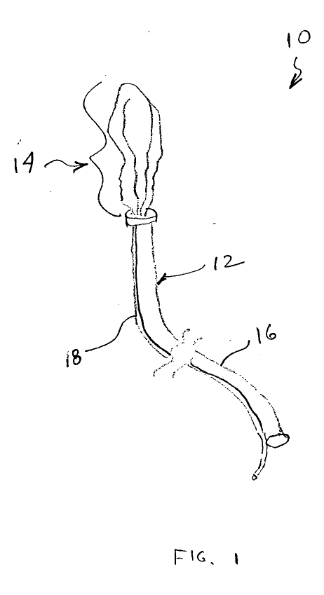

In one embodiment of the present invention shown in FIG. 1, a device that can be inserted into a patient to facilitate the clotting of blood is shown at reference numeral 10 and is hereinafter referred to as “device 10.” The device 10 comprises a flexible body portion 12 on which an expandable member 14 is located. As shown, the expandable member 14 is in an unexpanded state. In the illustrated embodiment, the expandable member 14 is a balloon, although other members (as described below) are within the scope of the present invention.

The flexible body portion 12 comprises a tube 16 through which a fluid can be passed to cause the expansion of the balloon 14 in the esophagus. A semi-flexible guide member 18 (e.g., a guide wire) may also be coextensive with the tube 16, incorporated into the structure of the tube, or attached to the tube. The guide member 18 is suitably rigid to enable the device 10 to be maneuvered through the throat and into the esophagus of a patient. A handle (not shown) may be attached to the flexible body portion 12 to facilitate the maneuvering of the device 10.

The balloon 14 is a non-porous bag-like element that is inflatable with a suitable fluid. Preferably, a saline solution is used to inflate the balloon 14, although gases such as air, nitrogen, and oxygen are also suitable if they meet the requisite sterility requirements. A syringe or hand pump may be used to pump the fluid to the balloon 14. The balloon 14 may be fabricated from any suitable material that is acceptable for surgical purposes, such materials including, but not being limited to, silicone elastomers, polyvinyl chlorides, polyethylenes, polyolefin copolymers, polyethylene terephthalates, and combinations of the foregoing.

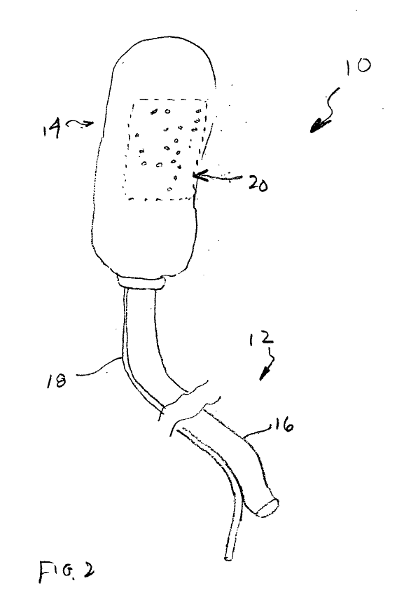

Referring now to FIG. 2, the device 10 is shown with the balloon 14 inflated. An outer surface of the balloon 14 includes at least one blood clotting material (e.g., zeolite or other molecular sieve material, diatomaceous earth, clay or other silica-based material, oxidized cellulose, carboxymethyl cellulose, salts of carboxymethyl cellulose, bioactive glass, biological hemostat, chitosan, combinations of the foregoing, or the like) incorporated therein or disposed thereon. The blood clotting material is preferably in particulate form, the particles being shown at 20. Although only a few particles 20 are shown, it should be understood that the surface of the balloon 14 is substantially covered with particles. The particles may be adhered directly to the surface of the balloon 14. Alternately, the particles 20 may be impregnated into any suitable substrate that can be attached to the outer surface of the balloon 14.

To adhere the particles directly to the surface of the balloon 14, a binder may be used. One suitable binder is chitosan, which also has hemostatic properties. The present invention is not limited to the use of chitosan as the binder, however, as other materials (e.g., polysaccharides, polyvinyl alcohol, guar gum, glycerol, gelatinized starches, cellulose (e.g., carboxymethyl cellulose), calcium alginate, combinations of the foregoing, and the like) are suitable for use as binders and are therefore within the scope of the present invention. In any embodiment, the material of the binder is biocompatible.

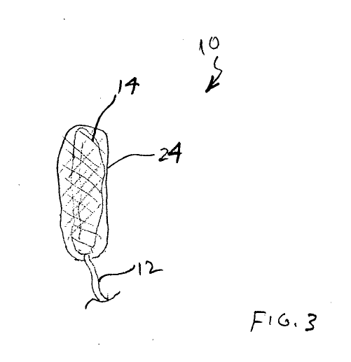

In its uninflated state, the balloon 14 may be encapsulated with a biocompatible wax coating 24 or similar substance, as is shown with reference to FIG. 3. The balloon 14 may be coated with such wax or similar substance to prevent the absorption of water by the blood clotting material. It is contemplated that upon insertion of the wax-covered (or other fluid) balloon 14 into the esophagus and the manipulation of the flexible body portion 12 to allow the balloon 14 to engage the bleed sites, the body heat of the patient would melt the wax, thereby exposing the balloon 14 and the incorporated blood clotting material to the blood.

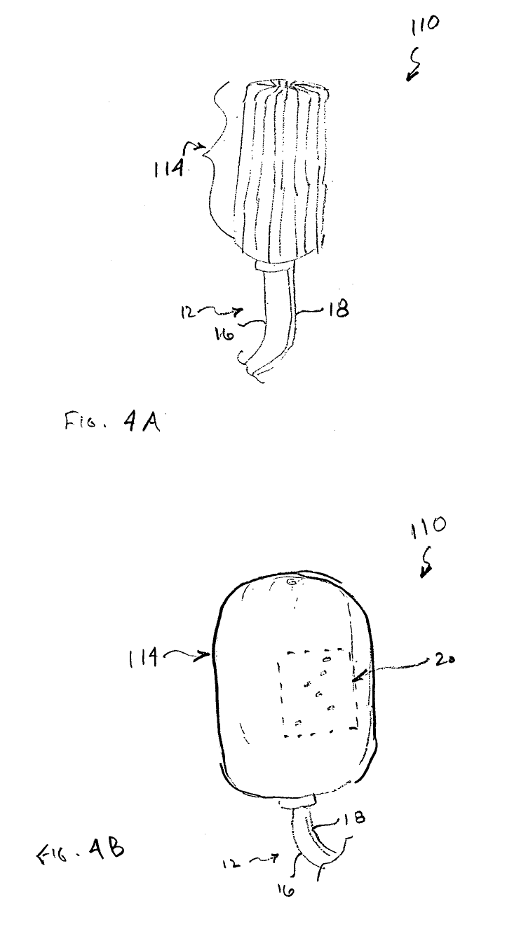

Referring now to FIGS. 4A and 4B, an alternate embodiment of a blood clotting device suitable for delivering blood clotting material to an esophageal varice is shown at 110. Device 110 comprises an expandable bellows 114 attached to a flexible body portion 12. As in the previously disclosed embodiment, the flexible body portion 12 comprises the tube 16 and the guide member 18. The bellows 114 is a pleated cylindrically-shaped member that, upon inflation, is capable of expanding. Inflation of the bellows 114 is effected in a manner similar to the inflatable balloon as described above. However, given the nature of the material from which the bellows 114 is fabricated and because the bellows is substantially rigid compared to the balloon, pressures required for the inflation of the bellows may be greater.

As is shown in FIG. 4B, once the bellows 114 is expanded, blood clotting particles 20 impregnated into or otherwise incorporated into the material of the bellows can engage the esophagus wall. The bellows 114 may be sized and configured such that upon expansion of the bellows, the curvature and contours of the peripheral surface of the bellows substantially correspond to the curvature and contours of the esophagus wall. The surfaces of the bellows 114 may be coated with a biocompatible wax to inhibit the absorption of moisture by the blood clotting particles 20 during insertion of the device 110. As with the balloon, it is within the scope of the present invention to impregnate or otherwise incorporate the blood clotting particles 20 into a suitable substrate that can be fixed to the bellows 114.

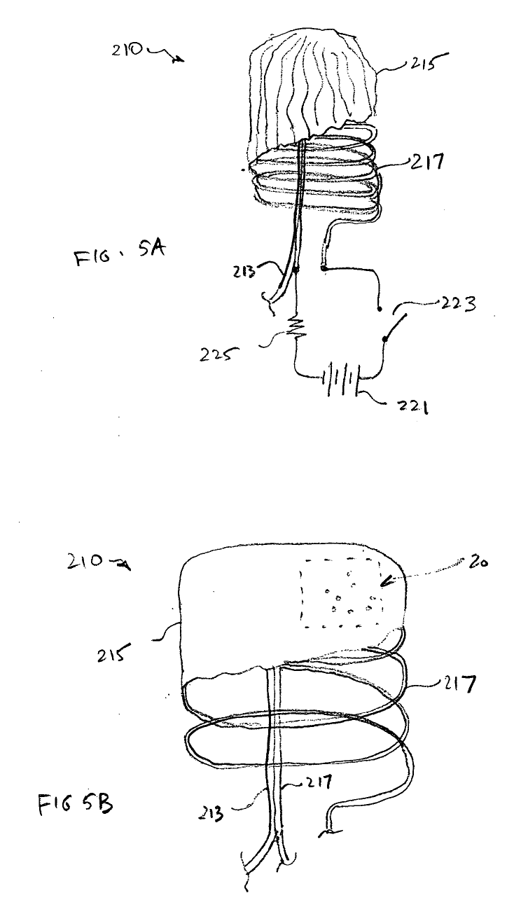

Referring now to FIGS. 5A and 5B, another alternate embodiment of a blood clotting device suitable for delivering blood clotting material to an esophageal varice is shown at 210. Device 210 comprises an expandable cylindrical member 214 having blood clotting particles incorporated or impregnated into or attached to an outer cover 215 disposed over an expandable spring 217. The expandable cylindrical member 214 may be attached to a flexible guide member 213 (e.g., a wire). When the device 210 is in its unexpanded state as shown in FIG. 5A, the outer cover 215 is bunched up, pleated, or folded in on itself in a lengthwise direction and the spring 217 is wound to be of a diameter to allow the device 210 to be inserted into the esophagus. When the device 110 is inserted into the esophagus of a patient, the spring 217 is selectively expandable to urge the outer cover 215 against the wall of the esophagus, thereby causing the blood clotting particles 20 to contact bleed sites on the esophagus wall.

Preferably, the spring 217 is fabricated from a shape memory alloy (“smart metal”) such as NITINOL. As is known, NITINOL is a nickel-titanium alloy that is capable of controlled deformation and reformation via a heating/cooling process. As used in the present invention, the NITINOL spring 217 is coil-shaped and deformed into an axially compressed state in preparation for use. Upon actuating by heating, the spring 217 expands axially to urge the outer cover 215 (and the blood clotting material) against the esophagus wall.

One method by which the NITINOL spring 217 may be expanded from its deformed and collapsed state, as is shown in FIG. 5A, to its expanded state, as is shown in FIG. 5B, is by the application of an electric current therethrough. The current may be applied from any suitable source, e.g., a direct current cell 221. A switch 223 controls the current flow. A resistor 225 or other device limits the current flow through the NITINOL to maintain a pre-selected temperature value to actuate the expansion. Once expanded, the blood clotting particles 20 can contact the bleed sites in the esophagus wall. Upon removing the current flow, the NITINOL cools and “remembers” its compressed shape, thereby pulling the outer cover 215 away from the esophagus wall. The device 210 can then be retracted from the patient.

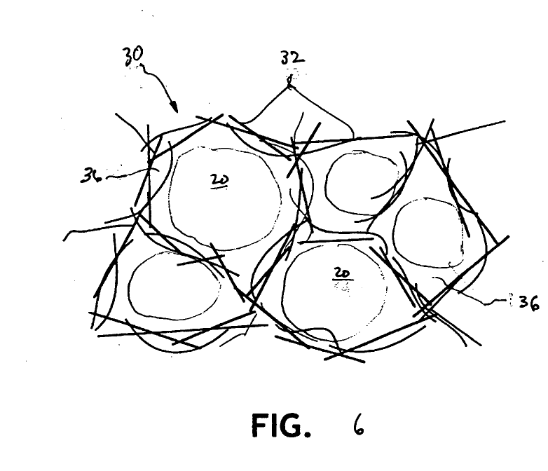

Referring now to FIG. 6, a suitable substrate which may form the fabric of the balloon, the bellows, or the outer cover (or be incorporated thereinto) is shown generally at 30. Substrate 30 is a porous web defined by interconnected fibers 32 such that the interconnection of the fibers retains the blood clotting material in particulate form therein. The blood clotting particles 20 can be incorporated into the porous web structure during formation of the substrate 30 or they can be impregnated into the finished substrate by conventional impregnation methods such as, but not limited to rolling. Various impregnation methods will produce differing loading levels for the blood clotting materials and also differing degrees of bonding of the blood clotting particles into the web.

The substrate 30 comprises the porous web and blood clotting particles 20 retained thereon by impregnation into interstices 36 defined by the fiber of the web material. As illustrated, the substrate 30 is planar, and only a few blood clotting particles 20 are shown for illustration purposes. While the blood clotting particles 20 have been shown and described as being retained in interstices 36 defined by the fiber of the web material, the present invention is not limited in this regard, as the particles can be adhesively or otherwise bonded to the substrate without departing from the broader aspects of the present invention.

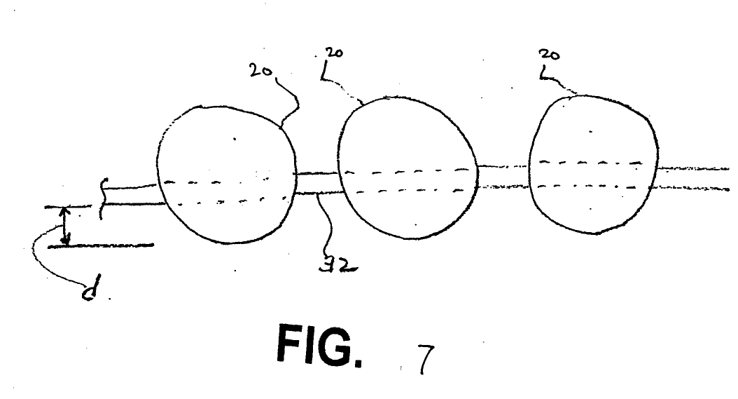

Referring now to FIG. 7, the interconnection of the fibers 32 defines the interstices such that the blood clotting particles 20 are retained but such that they extend out of the plane of the substrate 30 by a distance d. As such, the blood clotting particles 20 are allowed to come into direct contact with flowing blood. Because the substrate 30 may partially surround the blood clotting particles 20, portions of the particles may extend through the interstices. This allows the blood clotting material to directly contact tissue and thereby blood to which the blood clotting device is applied. Accordingly, blood emanating from the tissue contacts the blood clotting particles 20, and the liquid phase thereof is wicked into the material, thereby facilitating clotting. However, it is not a requirement of the present invention that the particles protrude out of the plane of the substrate.

With regard to the blood clotting particles 20, however, less particle surface area is available for contact with blood as particle size increases. Therefore, the rate of clotting can be controlled by varying the particle size. Furthermore, the adsorption of moisture (which also has an effect on the exothermic effects of zeolite when zeolite is used as the blood clotting material) can also be controlled.

Referring to both FIGS. 6 and 7, the fiber that defines the porous web of the substrate 30 may be paper, polymer, cloth, or any suitable natural or synthetic material. Paper fibers can be any cellulose-based material (e.g., wood, cotton, and the like). One particular type of paper that is useful in practicing the present invention is surgical grade kraft paper. Cellulose derivatives such as cellulose esters (e.g., cellulose acetate), cellulose ethers (e.g., methylcellulose), and cellulose nitrates (e.g., nitrocellulose) are also within the scope of the cellulose-based materials described herein.

Non-woven non-synthetic and synthetic cloth substrates can also be employed. Such substrates allow the underlying skin or tissue to “breathe” thereby providing for longer contact with damaged tissue since gaseous exchange can still take place. Non-woven non-synthetic and synthetic cloth substrates include, but are not limited to, TYVEK, GORTEX, and the like.

Polymer substrates can be any suitable polymeric material drawn into fiber form. Solid matrices are also useful where the blood clotting material is in particle form and the particles reside bound to the surface of a polymer sheet. Open-cell foam having porosity throughout the substrate to form a sponge structure is also desirable in some applications. The term “open-cell” as used herein shall be construed to mean that blood can pass into the cells to contact blood clotting material resident inside the cells. Polyethylene solid or open cell sponge material can form such a substrate wherein the blood clotting particles are bound in place but still allow intimate contact with the blood for desired clotting without leaving particles on the wound surface when the desired degree of clotting is achieved.

Synthetic polymeric plastics that can be used as substrates include, but are not limited to, MYLAR (polyethylene terephalate polyesters), polyethylene film, polypropylene film, polyethylene-polyamide laminated film, polyethylene-polyester laminated film, polypropylene-polyester laminated film, polyethylene-cellophane laminated film, and polyethylene-stretched polypropylene laminated film. Flexible, air permeable, high temperature resistant, bacteria-impermeable substrate material can preferably be made of non-woven polyester layers or polymeric fibrous materials such as polypropylene or polyester. The polyester can be located on either side of and bonded to a microporous membrane. Suitable polyesters include, but are not limited to REEMAY, which is available from BBA Fiberweb of Brentwood, Tenn., and VERATEC. Hydrophobic fluoropolymers such as microporous polytetrafluoroethylene; polyvinylfluoride, polyvinylidenefluoride, polychlorotrifluoroethylene, polyfluoroethylenepropylene, perfluoroalkoxyethylene and tetrafluoroethylene (TFE) copolymers; chlorotrifluoroethylene and ethylene copolymers; and TFE and ethylene copolymers are also suitable. However, the present invention is not limited in this regard.

In embodiments in which zeolites are utilized as the blood clotting material, the non-synthetic and synthetic substrates can be selected to tolerate the dehydration temperatures used to assure that the zeolite material has the desired level of water present to control any exothermic reaction and the temperature associated therewith. The dehydration temperature can be as low as 200 degrees Centigrade. Higher temperatures up to 400 degrees Centigrade reduce the time required to dehydrate the zeolite. However, the present invention is not limited in this regard as other temperatures and dehydration methods known to those skilled in the pertinent art to which the present invention pertains can be employed without departing from the broader aspects of the present invention.

In embodiments in which zeolite material is utilized as the blood clotting material, the control of the moisture content of the zeolite is related to its effectiveness. The preferred moisture content is between about 5 and about 25% by weight, more preferably between about 7 and about 19% by weight, and most preferably between about 10 and about 15% by weight. The moisture content of the zeolite can be adjusted by drying and then re-hydrating, or a combination of drying and re-hydrating, such that the zeolite has the desired specific moisture content. Alternatively, the zeolite may be fully saturated with water and subsequently dried to a specific water content. In the drying of the zeolite, the bound water is removed to allow the crystalline structure of the zeolite to remain intact. In the re-hydration of the zeolite, the most active adsorption sites are hydrated first and then less active sites are hydrated. As the degree of hydration of the zeolite increases, the heat of hydration decreases. More specifically, when the zeolite is applied to the blood, water in the blood is adsorbed by the zeolite. Upon adsorption of this water, heat is generated. At higher levels of hydration (hydration of the zeolite prior to its application to blood), less heat is generated when the zeolite is applied to blood. Thus, when the zeolite is applied to blood directly at a wound site, the amount of heat transferred to the tissue surrounding the wound site is reduced.

Whether the blood clotting material is zeolite, molecular sieve material, diatomaceous earth, clay, oxidized cellulose, or any other blood clotting material disclosed herein, the substrate 30 can be fixed to the expandable member by any suitable means. Suitable means of attaching the substrate 30 to the expandable member include, but are not limited to, welding, adhesive bonding, brazing, stitching, and the like.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A device for promoting the clotting of blood in a cavity, said device comprising:
a flexible body portion;
an expandable member located on said flexible body portion; and
a blood clotting material attached to said expandable member;
wherein when treating a bleeding wound, insertion of at least a portion of said blood clotting material into said cavity causes at least a portion of said blood clotting material to contact blood emanating from a bleed site.

2. The device of claim 1, wherein said blood clotting material is a zeolite.

3. The device of claim 2, wherein said zeolite comprises,
less than about 75% by weight of silicon oxide,
less than about 50% by weight of aluminum oxide,
less than about 30% by weight of sodium oxide, and
less than about 30% by weight of calcium oxide.

4. The device of claim 1, wherein said expandable member is a balloon.

5. The device of claim 4, wherein said flexible body portion is a tube through which said balloon can be inflated.

6. The device of claim 5, wherein said flexible body portion further comprises a guide member.

7. The device of claim 1, wherein said expandable member is a bellows.

8. The device of claim 7, wherein said flexible body portion is a tube through which said bellows can be inflated.

9. The device of claim 1, wherein said expandable member is expandable using a shape memory alloy.

10. The device of claim 9, wherein said shape memory alloy is a nickel-titanium wire.

11. The device of claim 9, wherein said shape memory alloy is configured as a coil spring that is compressed in an axial direction prior to actuation and expands upon actuation.

12. The device of claim 11, wherein said actuation is effected via heat from the passage of an electric current.

13. The device of claim 1, wherein the blood clotting material is disposed on a substrate that is attached to said expandable member.

14. The device of claim 1, wherein said device is used to promote the clotting of blood at an esophageal varice.

15. The device of claim 1, wherein said blood clotting material comprises diatomaceous earth.

16. The device of claim 1, wherein said blood clotting material comprises clay.

17. The device of claim 16, wherein said clay is selected from the group consisting of attapulgite, bentonite, kaolin, kaolinite, and combinations of the foregoing.

18. The device of claim 1, wherein said blood clotting material comprises oxidized cellulose.

19. The device of claim 1, wherein said blood clotting material is selected from the group consisting of carboxymethyl cellulose, salts of carboxymethyl cellulose, and combinations of the foregoing.

20. The device of claim 1, wherein said blood clotting material is selected from the group consisting of bioactive glass, biological hemostats, chitosan, and combinations of the foregoing.

21. A device for clotting blood in a tube-shaped body organ, said device comprising:
an elongated flexible member;
an expandable member located on said elongated flexible member; and
a blood clotting material disposed on an outer surface of said expandable member, said blood clotting material being effective for producing a clotting effect at a bleeding wound site.

22. The device of claim 21, wherein said blood clotting material is attached to a substrate and wherein said substrate is attached to said outer surface of said expandable member.

23. The device of claim 22, wherein said substrate comprises a material selected from the group of materials consisting of paper, cloth, polymers, and combinations of the foregoing.

24. The device of claim 22, wherein said substrate comprises a material selected from the group of materials consisting of non-woven natural cloth and non-woven synthetic cloth.

25. The device of claim 22, wherein said substrate comprises a material selected from the group of materials consisting of polyesters, polyethylene terephalate polyesters, polyethylene film, polypropylene film, polyethylene-polyamide laminated film, polyethylene-polyester laminated film, polypropylene-polyester laminated film, polyethylene-cellophane laminated film, polyethylene-stretched polypropylene laminated film, polytetrafluoroethylene, polyvinylfluoride, polyvinylidenefluoride, polychlorotrifluoroethylene, polyfluoroethylenepropylene, perfluoroalkoxyethylene, tetrafluoroethylene copolymers, chlorotrifluoroethylene and ethylene copolymers, tetrafluoroethylene and ethylene copolymers, and combinations of the foregoing.

26. The device of claim 22, wherein particles of said blood clotting material extend out of a plane of said substrate.

27. The device of claim 21, wherein said blood clotting material comprises a zeolite material comprising,
less than about 75% by weight of silicon oxide,
less than about 50% by weight of aluminum oxide,
less than about 30% by weight of sodium oxide, and
less than about 30% by weight of calcium oxide.

28. The device of claim 21, wherein said blood clotting material comprises diatomaceous earth.

29. The device of claim 21, wherein said blood clotting material comprises clay.

30. The device of claim 21, wherein said blood clotting material comprises oxidized cellulose.

31. The device of claim 21, wherein said blood clotting material is selected from the group consisting of carboxymethyl cellulose, salts of carboxymethyl cellulose, and combinations of the foregoing.

32. The device of claim 21, wherein said blood clotting material is selected from the group consisting of bioactive glass, biological hemostats, chitosan, and combinations of the foregoing.

33. A device for providing a therapy to an esophageal varice, said device comprising:
a flexible guide wire;
a flexible tube coextensively attached to said flexible guide wire;
an expandable balloon disposed in fluid communication with said flexible tube; and
a blood clotting material attached to said expandable balloon;
wherein when treating a bleed site associated with said esophageal varice, expansion of said expandable balloon causes said blood clotting material to contact said bleed site to effect the clotting of blood emanating from said bleed site.

34. The device of claim 33, wherein said expandable balloon is expandable using a pressurized gas.

35. The device of claim 33, wherein said expandable balloon is expandable using a pressurized saline solution.

36. The device of claim 33, wherein said expandable balloon is coated with a biocompatible wax prior to insertion into a patient, said biocompatible wax being meltable by body heat of said patient.

37. The device of claim 33, wherein said blood clotting material is a zeolite comprising,
less than about 75% by weight of silicon oxide,
less than about 50% by weight of aluminum oxide,
less than about 30% by weight of sodium oxide, and
less than about 30% by weight of calcium oxide.

38. The device of claim 33, wherein said blood clotting material comprises diatomaceous earth.

39. The device of claim 33, wherein said blood clotting material comprises a clay.

40. The device of claim 33, wherein said blood clotting material comprises oxidized cellulose.

41. The device of claim 33, wherein said blood clotting material is selected from the group consisting of carboxymethyl cellulose, salts of carboxymethyl cellulose, and combinations of the foregoing.

42. The device of claim 33, wherein said blood clotting material is selected from the group consisting of bioactive glass, biological hemostats, chitosan, and combinations of the foregoing.

43. A device for providing a therapy to an esophageal varice, said device comprising:
a flexible guide wire;
a flexible tube coextensively attached to said flexible guide wire;
an expandable bellows disposed in fluid communication with said flexible tube; and
a blood clotting material attached to said expandable bellows;
wherein when treating a bleed site associated with said esophageal varice, expansion of said expandable bellows causes said blood clotting material to contact said bleed site to effect the clotting of blood emanating from said bleed site.

44. The device of claim 43, wherein said blood clotting material is selected from the group consisting of zeolites, diatomaceous earth, clays, oxidized cellulose, bioactive glass, biological hemostats, chitosan, and combinations of the foregoing.

45. A device for providing a therapy to an esophageal varice, said device comprising:
a flexible guide wire;
an expandable cylindrical member;
a plurality of particles of a blood clotting material attached to an outer surface of said expandable cylindrical member; and
a shape memory alloy attached to said expandable cylindrical member, said shape memory alloy being configured to effect the expansion of said expandable cylindrical member;
wherein when treating a bleed site associated with said esophageal varice, expansion of said expandable cylindrical member causes said particles of said blood clotting material to contact said bleed site to effect the clotting of blood emanating from said bleed site.

46. The device of claim 45, wherein said shape memory alloy is configured to be a coil spring, said coil spring being expandable in an axial direction via the application of an electrical current thereto.

47. The device of claim 45, wherein said shape memory alloy is a nickel titanium alloy.

48. The device of claim 45, wherein said particles of said blood clotting material are attached to a substrate, and wherein said substrate is attached to said expandable cylindrical member.

49. The device of claim 45, wherein said blood clotting material is selected from the group consisting of zeolites, diatomaceous earth, clays, oxidized cellulose, bioactive glass, biological hemostats, chitosan, and combinations of the foregoing.

50. A method of clotting blood emanating from a tube-shaped internal organ, said method comprising the steps of:
providing a device having an expandable member having a blood clotting material attached to an outer surface thereof;
inserting said device into said tube-shaped internal organ of a patient;
positioning said device at a bleed site of said internal organ; and
expanding said expandable member to cause said blood clotting material to contact tissue of said bleed site.

51. The method of claim 50, wherein said step of positioning said device at said bleed site comprises maneuvering said device using a flexible guide wire.

52. The method of claim 50, further comprising a step of retracting said device from said internal organ of said patient.

53. The method of claim 50, wherein said step of expanding said expandable member comprises pressurizing a balloon with a fluid.

54. The method of claim 50, wherein said step of expanding said expandable member comprises pressurizing a bellows with a fluid.

55. The method of claim 50, wherein said step of expanding said expandable member comprises passing an electrical current through a wire fabricated from a shape memory alloy to effect an expansion of said wire.

56. The method of claim 50, wherein said blood clotting material is a zeolite.

57. The method of claim 50, wherein said blood clotting material is selected from the group consisting of diatomaceous earth, clays, oxidized cellulose, carboxymethyl cellulose, salts of carboxymethyl cellulose, bioactive glass, biological hemostats, chitosan, and combinations of the foregoing.

58. The method of claim 50, wherein said tube-shaped internal organ is an esophagus.

59. A method of providing blood clotting therapy to an esophageal varice, said method comprising the steps of:
providing a device having an expandable member having a blood clotting material attached to an outer surface thereof;
inserting said expandable member into an esophagus of a patient;
positioning said expandable member at a bleed site of said esophageal varice; and
expanding said expandable member to cause said blood clotting material to contact tissue of said esophageal varice.

60. The method of claim 59, wherein said step of positioning said expandable member at said bleed site comprises a step of maneuvering said device using a flexible guide wire.

61. The method of claim 59, further comprising a step of retracting said device from said esophagus of said patient.

62. The method of claim 59, wherein said step of expanding said expandable member comprises inflating said expandable member with a fluid or expanding a shape memory alloy using an electrical current.

63. The method of claim 59, wherein said blood clotting material comprises a zeolite.

64. The method of claim 59, wherein said blood clotting material comprises diatomaceous earth.

65. The method of claim 59, wherein said blood clotting material comprises clay.

66. The method of claim 59, wherein said blood clotting material comprises oxidized cellulose.

67. The method of claim 59, wherein said blood clotting material is selected from the group consisting of carboxymethyl cellulose, salts of carboxymethyl cellulose, and combinations of the foregoing.

68. The method of claim 59, wherein said blood clotting material is selected from the group consisting of bioactive glass, biological hemostats, chitosan, and combinations of the foregoing.
